(19) United States
(12) Patent Application Publication
(10) Pub. No.: US 2004/0215234 A1
(43) Pub. Date: Oct. 28, 2004

(54) Flexible baby teether feeding stick

(76) Inventor: Laurie Wynne McCardell, Casper, WY (US)

Correspondence Address: Laurie Wynne McCardell, 1045 Horseshoe Road, Casper, WY, 82604, US

(21) Appl. No.: 10/423,298

(22) Filed: Apr. 28, 2003

(51) Int. Cl. A61J 17/00; A61B 17/00

(52) U.S. Cl. 606/235; 606/234

(57) ABSTRACT

The Flexible Baby Feeding Stick is a new method of feeding the 4 to 18+ month-old baby. The stick is approximately 4 inches long with two approximately ¾ inch diameter balls on either end. The entire surface is covered with a raised bumpy pattern which holds baby food, as well as massaging the gums of the teething infant. The baby can hold either end or the middle of the stick, and suck off the food from the stick. There is no “up” or “down” on the stick as there is on a spoon. Therefore, the baby will not become frustrated by having food slide off the stick (as it does on a spoon) before they get the stick to their mouth. The feeding stick is a safe tool to encourage a baby to self-feed.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF INVENTION

This invention pertains to the messy process of teaching an infant to feed his or herself. There are a number of types of spoons on the market, many of them designed for the teething infant. These spoons have soft edges and are made of plastic, or rubber-wrapped metal, and come in various shapes and sizes. All these spoons must be held correctly by the infant (or the person feeding the child) so that the food does not drop off.

### BRIEF SUMMARY OF THE INVENTION

This baby feeding stick is designed for the baby (approximately 4 months to 18 months old) to feed his or herself. The baby would grip the center of the rubbery and somewhat flexible feeding stick, and chew or suck on either rounded end of the stick to feed his or herself. For a younger (4 to 12 month baby) a care-giver could dip the feeding stick into a jar of baby food or other moist food of the appropriate consistency. The baby would then suck the food off either end. An older baby would be able to dip the stick in the food him or herself, and then self-feed.

### BRIEF DESCRIPTION OF THE SEVERAL VIEWS OF THE DRAWING

FIG. 1A is a side view of the feeding stick. This view would look the same if you rolled the stick on a table top in order to see the other portions of the stick.

FIGS. 1B and 1C are both head-on views of the stick from either end.

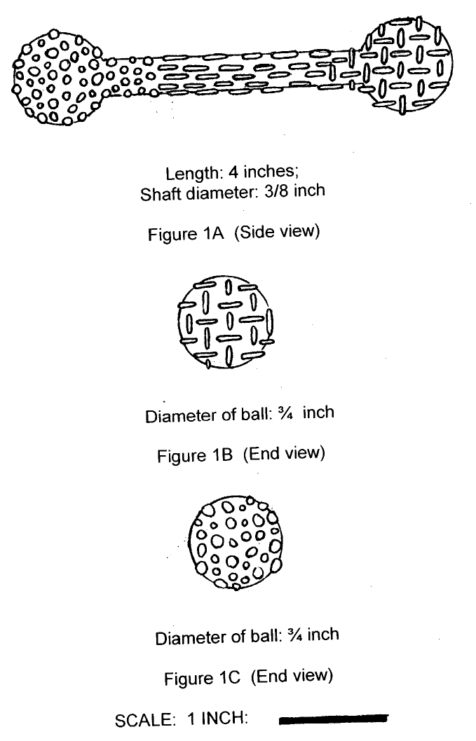

### DETAILED DESCRIPTION OF THE INVENTION

The flexible baby feeding stick is designed to:

1) keep the baby occupied during meals by having something easy to hold and chew on;

2) Provide an introduction to different tastes and smells without stuffing too much food at once into the baby's mouth;

3) Eliminate the normal frustration for the self-feeding baby in having the food drop off of a baby spoon when the spoon is not held upright;

4) Massage the baby's gums and help with teething crankiness. The stick's surface is covered with a raised pattern approximately 1/16^{th} inch high, that will both hold some food, and massage the baby's gums.

5) Provide a baby-friendly feeding item that the baby can grasp and use; (Most babies tend to grab onto a spoon that is being used by someone else to feed them. The result of the “spoon grab” creates more mess and further frustration on the part of both baby and care-giver) p1 6) Allow the baby to eat/suck slowly without choking on a big spoonful of food;

7) Bend in the middle with moderate pressure, making it more interesting for the baby to use;

8) Be made out of a molded rubber/nylon substance that is dishwasher safe.

In summary, the feeding stick is an alternative to a baby spoon for the infant. It helps the baby to self-feed, thereby reducing frustration by both baby and care-giver during mealtimes. It is grasped easily by the baby, fits nicely in their hand, and provides a teething surface that also holds the right amount of food so that they can eat in small, safe portions.

1) What I claim as my invention is the flexible baby feeding stick with a shaft diameter of approximately ⅜ inch, with two spheres on either end of the stick which are approximately ¾ of an inch in diameter, and which measures approximately 4 inches from one end to the other.

2) I claim a bumpy patterned surface over the entire feeding stick that massages the gums of the teething infant.

3) I claim a bumpy surface on the feeding stick (approximately 1/16^{th} inch raised bumps) that holds enough food (but not too much food) for the beginning self-feeding infant so that they can eat in small safe portions.
